(19) United States
(12) Patent Application Publication
(10) Pub. No.: US 2016/0317467 A1
(43) Pub. Date: Nov. 3, 2016

(54) METHOD OF TREATING OR PREVENTING INFECTION

(71) Applicant: DAA, INC., Huntington Station, NY (US)

(72) Inventor: Abby ARONOWITZ, Huntington Station, NY (US)

(21) Appl. No.: 15/209,365

(22) Filed: Jul. 13, 2016

Related U.S. Application Data
(63) Continuation of application No. 14/287,165, filed on May 26, 2014.

(51) Int. Cl. A61K 31/047; A61K 33/30; A61K 9/00

(52) U.S. Cl. CPC A61K 31/047; A61K 9/0031; A61K 9/0046; A61K 33/30

(57) ABSTRACT

A method of treating and preventing infection using an effective concentration of xylitol in a pharmaceutical composition.

### CROSS-REFERENCE TO RELATED APPLICATIONS

This Continuation application claims priority to and the benefit of U.S. patent application Ser. No. 14/287,165, filed May 26, 2014, the disclosures of which are incorporated herein in their entirety by reference.

### FIELD OF THE INVENTION

The field of the invention is anti-microbial therapy.

### BACKGROUND

In U.S. Pat. No. 8,680,263, Kozlowski, et al. describe the use of compounds including polysaccharaides to form conjugates which can be used for drug delivery and purification. This is based upon the fact that polar organic functional groups can form weak interactions with drug molecules, which often have polar sections as well.

### OBJECTS OF THE INVENTION

One object of the invention is to develop a novel antimicrobial. Another object of the invention is to develop a preventative treatment for airborne pathogens to arrest epidemics. Yet another object of the invention is to develop a pathogen mitigating enema. Another object of the invention is to develop a pathogen mitigating solution to target bacteria in the ears, lungs, throat, colon, anus, and bladder. Another object of the invention is to develop delivery systems which would target specific, localized areas, such as an enema for the colon, peri-anal wipe for the anus, syringe for bladders, ear dropper for ears, inhaler or nebulizer for lungs, and spritz or pre-moistened towelette for cleaning surfaces.

Another object is gargle for the throat, and a product combining zinc with xylitol, to minimize upper respiratory symptoms.

### SUMMARY

The invention is the use of xylitol as a microbial infection inhibitor for non-sinus, non-oral, treatments.

### DETAILED DESCRIPTION OF THE INVENTION

Xylitol is a pentose polyol which has ligand-molecule interactions with many binding site proteins and polysaccharides found in pathogen attachment mechanisms. A solution of xylitol will therefore form complexes with the binding sites and inhibit microbial attachment. Attachment is the first step in many human pathogen infections by microbes.

### EXAMPLES OF THE INVENTION

In one preferred embodiment of the invention, a solution of 10% xylitol in water w/w is sprayed into the ambient air via a mister during an outbreak of airborne microbe borne infection epidemic.

In another preferred embodiment, the solution, ranging from 1%-95% xylitol in a carrier is injected into the lower colon via an enema to reduce colon mucus microbial attachment.

In yet another preferred embodiment the solution of xylitol is inhaled into the lungs via an atomizer to prevent or neutralize lung infections.

In yet another preferred embodiment, a xylitol paste with 95% xylitol w/w and 5% w/w alcohol is applied via application tube to the anus.

In yet another preferred embodiment, a 8% w/w xylitol in physiological buffered solution is introduced to the bladder via a catheter in a syringe to prevent bladder infection. A 1% to 95% solution of xylitol in a carrier is also contemplated.

In yet another preferred embodiment, a solution of 10% w/w xylitol is blended with a variety of oils in an emulsion for use as a skin cream cosmetic with antimicrobial properties.

In yet another preferred embodiment, 10% w xylitol is mixed with a ethanol-water solution for use as hand sanitizer.

What is claimed is:

1. A method to treat or prevent infection of the colon comprising: administering an anti-microbial adhesion inhibitor pharmaceutical composition for localized application via an enema solution to prevent and/or treat infections of the epithelial and mucosal tissues originating from pathogens from the list consisting of viruses, protozoans, gram negative and grain positive bacteria, and said composition comprising an active agent of xylitol present in a concentration of 1% to 95% by weight in a physiologically buffered acceptable carrier.

2. A method to treat or prevent infection of the colon as recited in claim 1 wherein said composition is used to treat pathogens from the list consisting of E. Coli, staphylococcus, streptococcus pneumonia, and Clostridium difficile in a pharmaceutical composition.

3. A method to treat or prevent infection of the colon as recited in claim 1, wherein said acceptable carrier is water.

4. A method to treat or prevent infection of the colon as recited in claim 1, wherein said acceptable carrier is a water and alcohol solution.

5. A method to treat or prevent infection using a pharmaceutical composition as recited in claim 1 wherein said xylitol is present in a concentration between 8% and 10% by weight in a pharmaceutical composition.

6. A method to treat or prevent infection as recited in claim 1 wherein said composition further comprises zinc salts.

7. A method to treat or prevent infection of the bladder comprising: administering an anti-microbial adhesion inhibitor pharmaceutical composition for localized application via a bladder irrigation solution to prevent and/or treat infections of the epithelial and mucosal tissues originating from pathogens from the list consisting of viruses, protozoans, gram negative and gram positive bacteria, and said composition comprising an active agent of xylitol present in a concentration of 1% to 95% by weight in a physiologically buffered acceptable carrier.

8. A method of treatment to treat or prevent infection using an antimicrobial adhesion inhibitor pharmaceutical composition for localized application in preventing and/or treating infections of the epithelial and mucosal tissues of a mammal's ear originating from pathogens from the list consisting of viruses, protozoans, gram negative and gram positive bacteria, and said composition consisting essentially of an active agent of xylitol present in a concentration of 1% to 95% by weight in an acceptable carrier in a pharmaceutical composition.

9. A method to treat or prevent infection of the bladder using a pharmaceutical composition as recited in claim 7, wherein said acceptable carrier is water.

10. A method to treat or prevent infection of the bladder using a pharmaceutical composition as recited in claim 7, wherein said acceptable carrier is a water and alcohol solution.

11. A method to treat or prevent infection using a pharmaceutical composition as recited in claim 7 wherein said xylitol is present in a concentration between 8% and 10% by weight in a pharmaceutical composition.

12. A method to treat or prevent infection as recited in claim 1 wherein said composition further comprises zinc salts.
